(19) United States
(12) Patent Application Publication
(10) Pub. No.: US 2009/0177028 A1
(43) Pub. Date: Jul. 9, 2009

(54) Non-blood contact cardiac compression device, for augmentation of cardiac function by timed cyclic tensioning of elastic cords in an epicardial location

(76) Inventor: Anthony John White, Venice, CA (US)

Correspondence Address: Anthony John White, 829 N. Sweetzer Ave, W. Hollywood, CA, 90069, US

(21) Appl. No.: 12/006,570

(22) Filed: Jan. 4, 2008

(51) Int. Cl. A61F 2/02

(52) U.S. Cl. 600/37

(57) ABSTRACT

One embodiment of a device to augment the pumping function of a weakened heart. A set of connectors with elastic properties are positioned immediately proximate to the outer surface of the heart. Each of the elastic connectors is attached at one end to a circumferential band firmly attached to the exterior surface of the heart at around the level of the atrioventricular groove, an anatomical feature of the heart. At the other end, each of said elastic connectors is attached to a cap attached firmly to the heart exterior of the heart at the anatomical apex. A mechanism places the elastic connectors alternatively under tension (stretch) then shortening (relaxation). When the elastic connectors are stretched they draw the apex and circumferential band together, resulting in external compression of the heart. This movement causes the internal volume of the cardiac ventricles to be reduced, thereby encouraging expulsion of the blood contents of the ventricles to be expelled. This action will be timed so as to augment the natural cardiac contraction, and thereby improve its pumping function.

### FIELD OF THE INVENTION

This invention generally relates to physical devices for the treatment of human heart failure, and more specifically to assisting the natural heart's function by intermittent stretching of a number of longitudinal elastic fibres positioned in immediate proximity to the external surface of the heart, carefully timed to the heart's natural beating.

### BACKGROUND OF THE FIELD

Heart failure is also commonly referred to as congestive cardiac failure (CCF). This is a common clinical syndrome, in which the cardiac output (measured in liters per minute) is inadequate to provide enough blood flow to the body's tissues to meet their demand for oxygen and nutrients.

There are many causes of cardiac failure, the commonest of which are hypertension, coronary artery disease and dilated cardiomyopathy (the last of these categories is an intrinsic weakness of heart muscle contraction). Another broad way to sub-divide cardiac failure is as systolic heart failure (weakened contractile function), or diastolic heart failure (limitation of ventricular filling because the heart chamber is stiff). When cardiac output is so low that an adequately high blood pressure cannot even be maintained, the scenario is known as cardiogenic shock; this is the most severe type of heart failure, and is associated with multi-organ failure, and 90% probability of incipient death.

Symptoms of cardiac failure include shortness of breath (especially with physical exertion or lying flat), accumulation of fluid around the ankles and/or in the lungs and fatigue. The disorder is common, especially in elderly individuals, with over half a million new cases diagnosed annually in the USA. There are an estimated 22 million individuals living with cardiac failure in the world. The incidence of cardiac failure is projected to rise due to increasing average age of the population, as well as improved survival after heart attack, such that more individuals are living with a damaged heart. After a diagnosis of cardiac failure is made in an individual, the probability of them being alive in five years is only about 50%. The mode of death is most often progressive heart failure (“pump failure”) or sudden death due to heart rhythm disturbance.

The present invention would be likely to have a therapeutic role in individuals at the severe end of the spectrum of cardiac failure. Cardiac failure symptoms are often classified according to the New York Heart Association (NYHA) scale. Using this scale, persons experiencing class III symptoms (breathless on minor exertion) or Class IV symptoms (breathless at rest—essentially bed bound) or cardiogenic shock are the likely groups to benefit from this invention. Initial trials would test the invention as a “bridge” to allow survival until either cardiac transplantation or recovery of cardiac function. If proven in this setting, the invention may then some day be tested as a stand-alone therapeutic procedure, with no plan for eventual cardiac transplantation. Expert discussion within the field refers to this second type of use as “destination therapy” for severe heart failure.

### BACKGROUND OF PRIOR ART

Effective medical and surgical treatments have been developed for cardiac failure, and can be characterised as:

1. Treatments aimed at the underlying cause: This includes:
a. coronary artery bypass surgery or coronary artery stenting, to improve blood supply to heart muscle if this is the root cause. Control of high cholesterol, tobacco exposure and diabetes are equally important if coronary disease is the underlying cause.
b. Treatment of high blood pressure.

2. Drug treatments. Proven drug treatments for heart failure include:
a. ACE inhibitors, for example the drugs ramipril, perindopril and enalapril
b. Beta-blockers, for example the drugs carvedilol, bisoprolol and long acting metoprolol.
c. Aldosterone receptor blockers, for example the drugs spironolactone and eplerenone.
d. Diuretics to promote fluid excretion by the kidneys. Although these do not extend the life-span, they are effective in relieving symptoms of ankle swelling and shortness of breath.

3. Devices:
a. Bi-ventricular pacemaker: this device alters the timing of contraction in various parts of the heart, thereby improving efficiency of the “pump”. This therapy is also referred to as cardiac resynchronization therapy (CRT). (Abraham, W. T., et al., Cardiac resynchronization in chronic heart failure. New England Journal of Medicine, 2002. 346: p 1845).
b. Automated implantable cardiac defibrillator. These devices are designed to detect what would have been a fatal heart rhythm disturbance and deliver a shock to save the patient's life in that situation. (Bristow, M. R., et al., Cardiac-resynchronization therapy with or without an implantable defibrillator in advanced chronic heart failure. New England Journal of Medicine, 2004. 350: p 2140 and Moss, A. J., et al., Prophylactic implantation of a defibrillator in patients with myocardial infarciton and reduced ejection fraction. New England Journal of Medicine, 2002. 346: p 877).

Unfortunately, although the above treatments improve the situation, many individuals remain symptomatic and high rates of death persist.

A final category of treatment for heart failure is cardiac transplantation. A successful cardiac transplant is very often truly life-changing for an individual, with a return to an active and productive life. Around 3,000 cardiac transplants are performed globally, annually. This number is unlikely to increase to any great extent, due to a limited supply of donor hearts, and the enormous co-ordinated societal effort required to organize timely supply of organs. Hence, although on an individual level, the benefit can be enormous, from a wider perspective this solution is only ever going to benefit an exceedingly small proportion of the large number of individuals afflicted by heart failure.

Hence there is an unequivocal clinical, economic and humanitarian need for advances in the treatment of cardiac failure.

It is against this background, that there has been great interest in developing a “mechanical” solution to the problem. In other words, a device to assist, augment or completely take over the pumping function of the failing heart.

### Mechanical Circulatory Support

Development of artificial intra-thoracic circulatory pumps began over 40 years ago, (Hall, C. W., et al., Development of Artificial Intrathoracic Circulatory Pumps. Am J Surg, 1964. 108: p685). The first artificial heart implant was performed in 1969 (Cooley, D. A., et al., Orthotopic cardiac prosthesis for two-staged cardiac replacement. Am J Cardiol, 1969. 24: p. 723), and kept the patient alive for 64 hours until a transplant became available.

Two important conceptual developments in the field have taken place recently.

The first concept followed logically from the results of the REMATCH clinical trial. (Rose, E. A., et al., Long-term mechanical left ventricular assistance for end-stage heart failure. N Engl J Med, 2001. 345: p. 1435). In this trial, the survival of patients with severe heart failure was compared between those with left ventricular assist device (LVAD) insertion, and those with standard medical therapy. The LVAD group had improved survival, which led to support for the concept of an LVAD as so called “destination therapy” in its own right, and not just as a strategy to prolong life until a cardiac transplant became available.

The second concept is that if cardiac function is mechanically supported to allow survival for a period of months, cardiac function will often improve (Levin, H. R., et al., Reversal of chronic ventricular dilation in patients with end-stage cardiomyopathy by prolonged mechanical unloading. Circulation, 1995. 91: p. 2717), to the point where the device assisting cardiac function may be able to be removed (Birks, E. J., et al., Left ventricular assist device and drug therapy for the reversal of heart failure. New England Journal of Medicine, 2006. 355: p. 1873).

Mechanical pumps or Ventricular Assist Devices (VADs) are pumps which augment the cardiac output. They can provide pulsatile or continuous flow, and can be implanted internally or external to the body, with pipes taking blood out of, and back into the circulation.

Examples of these devices include:
Thoratec® VAD (Thoratec, Inc): can be used for LV, RV or biventricular assistance. It is approved for use as a “bridge to transplant” or circulatory support after cardiac surgery.
HeartMate VAD (Thoratec, Inc): This was the device used in the REMATCH trial, and has been approved by the FDA as a “destination therapy” in specific circumstances. (Severe heart failure, expected to live less than 2 years).
Novacor® VAD (World Heart Corporation) provides pulsatile LV assistance, and has been implanted in over 1700 individuals. The clinical trial called RELIANT has been started to compare the Novacor with the HeartMate to evaluate suitability of Novacor as destination therapy for end-stage cardiac failure.
Ventrassist (Ventracor Ltd). This implantable LVAD was designed and developed in Australia. Its mechanism includes an “impeller”, a bearing-less set of blades rotated magnetically. (Patent international publication number WO2005/032620 A1). It provide continuous, rather than pulsatile flow.
Arrow Lionheart-2000 (Arrow International). This is an experimental device. Its great potential advantage is that it is fully implanted. No lines breach the skin once implanted. (Mehta, S. M., et al., The LionHeart LVD-2000: a completely implanted left ventricular assist device for chronic circulatory support. Annals of Thoracic Surgery, 2001. 71. p. S156)

Other intra-corporeal devices in various stages of development include the AbioCor (AbioMed), Jarvik 2000, MicroMed Debakey and Kriton VADs.

The VADs described above have the pump fully implanted within the body. Those that follow have the pump external to the body.
TandemHeart (CardiacAssist, Inc.) This is approved for short term use (<6 hours) for circulatory support. It is placed percutaneously, and draws blood out of the left atrium, through an extracorporeal pump, and returns it to one or both femoral arteries.
Impella Recover (Impella Cardiosystem AG)¹⁵ is a catheter based system that sucks blood from the left ventricle at up to 5 litres per minute and delivers in into the aorta. (Jermann, M. J., et al., Initial experience with miniature axial flow ventricular assist devices for postcardiotomy heart failure. Annals of Thoracic Surgery, 2004. 77. p. 1642)
AbioMed BVS 5000 (AbioMed) is a LVAD or biventricular support device available for short term use (7-10 days) post cardiac surgery whist awaiting recovery of cardiac function.

Overall, LVADs have been shown to improve exercise tolerance, and survival compared to medical therapy, and may allow survival until a transplant is available or the heart recovers.

However, major problems are inherent to mechanical LVADs, including:
Thrombosis (blood clotting) resulting from contact of blood with metallic and polyurethane components of the device. Clot can also embolize (travel) from the device and cause stroke.
Infection, resulting from implantation of a large foreign body and breaches in the skin integrity by tubes or cables that exit the body.
Bleeding complications resulting from the strong blood thinning medications required to prevent thrombosis in the devices.
Device failure.

An enormous advantage of the present embodiment over mechanical LVADs is that it avoids direct contact with blood, thereby eliminating the problem of clot formation on the device surface, which is a major limitation when blood flows through LVAD devices

### Passive Cardiac Constraint Devices

The concept of passive constraint of the heart arose out of old studies which investigated wrapping the heart in skeletal muscle (“lattisimus dorsi wrap”), with the idea of stimulating the muscle to contract in time with the heart, thereby augmenting cardiac contraction (Patel, H. J., et al., Dynamic cardiomyoplasty: its chronic and acute effects on the failing heart. Journal of Thoracic and Cardiovascular Surgery, 1997. 114: p. 169). The method was not very good at augmenting contraction, but some patients still seemed to benefit. The reason for this may have been that the wrap caused a passive limitation to the cardiac dilatation that accompanies end-stage heart failure.

Many such devices have been granted patents. One is the Acorn passive constraint, which is a snug bag for the heart which limits its enlargement, thereby reducing ventricular wall stress (Alferness in U.S. Pat. No. 7,166,071 (2007), U.S. Pat. No. 7,163,507 (2007), U.S. Pat. No. 7,025,719 (2006) and U.S. Pat. No. 5,702,343, and Girard et al in U.S. Pat. No. 6,951,534 (2005) amongst others). Other passive constraint devices, designed to limit ventricular wall tension include those of Lau et al in U.S. Pat. Nos. 7,189,202 and 7,174,896 (2007) and U.S. Pat. No. 7,097,611 (2006), U.S. Pat. No. 7,276,021 (2007) amongst others. Other devices also have pacing or defibrillation capability such as Lau et al in U.S. Pat. Nos. 7,187,984, 7,164,952 and 7,158,839 (2007) and U.S. Pat. No. 7,155,295 (2006) amongst others.

Two other similar innovations being tested, involving bars external to the left ventricle which indent it, and change the cross section of the left ventricle from one dilated circle, to an “8” shaped cross-section of two circle with smaller diameters, thus reducing ventricular wall stress. These devices are the Myocor Myosplint device and the CardioClasp device (U.S. Pat. No. 6,190,408). An incremental improvement upon the CardioClasp has also been patented (International Publication Number WO2004/010875: Cyclic Device for restructuring heart chamber geometry) whereby energy from cardiac filling is stored during cardiac filling, then that energy is reapplied to the system during cardiac contraction.

Other patents awarded for passive cardiac constraint devices have included U.S. Pat. No. 6,050,936 to C. J. Schweich. “Heart wall tension reduction apparatus.” (Myocor Inc) and U.S. Pat. No. 5,800,528 to D. M. Lederman “Passive girdle for heart ventricle for therapeutic aid to patients having ventricular dilatation” (Abiomed R&D, Inc)

The current embodiment is similar to passive constraint devices, only in that the device is closely in apposition to the external surface of the heart. However, the currently described invention is superior to passive constraint devices in the following ways:
It does not restrict filling of the ventricles with blood at the appropriate time of the cardiac cycle, because the elastic straps are made completely loose during diastole, thus imposing no limitation of ventricular expansion during the filling phase of the cardiac cycle
Rather than only passively limiting dilatation of the cardiac chambers as they fail, the current invention actively provides additional energy to augment contraction during each heart beat.

### External Cardiac Compression

The present invention falls within the general category of external cardiac compression device.

Experiments with isolated animal hearts have provided proof that external compression of a beating heart, timed to co-ordinate with the heart's own contraction (systole), is effective in increasing stroke volume, without increasing cardiac work or comprising coronary flow (Artrip, J. H., et al., Hemodynamic effects of direct biventricular compression studied in isovolumic and ejecting isolated canine hearts. Circulation. 1999. 99: p. 2177).

Melvin (U.S. Pat. No. 6,988,982 (2006) and International publication number WO2004/016159) claims a method for assisting the operation of the natural heart comprising positioning an actuator element proximate to the heart and operating the actuator element to act on a heart wall portion to effect a change in the shape of the heart. U.S. Pat. No. 6,988,982 is similar to the present invention only because it describes a device closely approximated to the external wall of the heart. However the present invention is very different from the device described in U.S. Pat. No. 6,988,982 in the following ways:
The present invention describes the cyclical stretching of connectors with elastic properties to augment cardiac function, not the solid shape limiting elements with hinged links described in U.S. Pat. No. 6,988,982
In the present invention a circumferential band is anchored firmly to the cardiac tissue at or around the level of the atrio-ventricular groove, such that the elastic fibres have an anchored attachment point both here are at the cardiac apex
In the present invention the activator mechanism acts to alternately stretch and relax the elastic connectors which is not claimed in U.S. Pat. No. 6,988,982

A number of patents describe implantatable heart compression devices that involve various types of flexible fluid-filled chamber or bladder placed around the heart that are intermittently inflated to compress the heart, thereby encouraging ejection of blood from the ventricles. For example, Coleman et al, in U.S. Pat. No. 7,118,525 (2006) describes an implanted cyclic cardiac compression device that utilises fluid filled chambers that intermittently inflate and deflate to augment cardiac function. Milbocker, in U.S. Pat. No. 6,602,182 (2003), U.S. Pat. No. 6,616,596 (2003), U.S. Pat. No. 6,547,716 (2003) and U.S. Pat. No. 6,540,659 (2003) describes an implantable set of fluid filled chambers that wrap around the heart. Other types of devices that use this general type of design (arrangement of fluid filled bladder(s) around the heart) are described by Herrero in U.S. Pat. No. 6,387,042 (2002); Easterbrook III in U.S. Pat. No. 6,238,334 (2001); Schiff in U.S. Pat. No. 3,587,567; Heid in U.S. Pat. No. 3,371,662; Paravicini in U.S. Pat. No. 4,536,893 (1985); Freeman in U.S. Pat. No. 4,448,190 (1984); Goetz in U.S. Pat. No. 4,048,990 (1977); Ascrican in U.S. Pat. No. 4,192,293; Bolie in U.S. Pat. No. 3,233,607; and Kline in U.S. Pat. No. 3,279,464. The present invention is distinct from the above patents because it describes the cyclical stretching of connectors with elastic properties to augment cardiac function, not the cyclical inflation of fluid filled bladders around the heart.

Shahinpoor in U.S. Pat. No. 6,464,655 (2002) and in U.S. Pat. No. 7,198,594 (2007) describes devices that utilize soft robotic fingers to cyclically compress the heart. The present invention is distinct these patents because it describes the cyclical stretching of connectors with elastic properties to augment cardiac function, not the use of robotic fingers to achieve this aim.

Finally, another type of ventricular assist device has been described that utilises intermittent tightening then loosening of an external cardiac strap(s), by Freeman and Maynard in U.S. Pat. No. 4,304,225 (1981) and Heilman et al in U.S. Pat. No. 5,558,617 (1996), U.S. Pat. No. 5,383,840 (1995) and U.S. Pat. No. 4,925,443 (1990). One of the novel features of the current invention compared to the four patents just mentioned is that several components of the device are firmly attached to the external surface of the heart in the current invention; namely the circumferential component anchored firmly to the outer surface of the heart at approximately the level of the atrio-ventricular groove, and the cardiac cap component attached firmly to the external aspect of the apex of the heart. The U.S. patents referred to earlier in this paragraph describe external straps applied to the external surface of the heart, but not firmly attached to the organ.

Another device described is the prior art is “Cyclic Device for restructuring heart chamber geometry” (International Publication Number WO2004/010875). The presently described invention is superior because additional energy is applied to the failing heart during each heart beat, whereas the “Cyclic Device for restructuring heart chamber geometry” merely seems to harvest cardiac energy during diastole, store it as potential energy, and then re-deploy this energy during systole. That is, there is no new energy employed by the system to the contraction of the heart.

### SUMMARY OF THE INVENTION

This embodiment addresses the objective of augmenting the pumping function of the heart by means of connectors with elastic properties, positioned immediately proximate to the outer surface of the heart. Each of the elastic connectors is attached at one end to a circumferential band firmly attached to the heart, and at the other end to a component attached firmly to the heart at the anatomical apex of the heart. A mechanism exists to place the elastic connectors alternatively under tension (stretch) then being allowed to elongate (relax). When the elastic connectors are stretched they will have the natural characteristic to draw the apex and circumferential band together, resulting in external compression of the heart. This movement will cause the internal volume of the cardiac ventricles to be reduced, thereby encouraging expulsion of the blood contents of the ventricles to be expelled. This action will be timed so as to augment the natural cardiac contraction.

### BRIEF DESCRIPTION OF THE DRAWINGS

The accompanying drawings, which constitute a part of this specification, illustrate embodiments of the invention and aim to illustrate the principles of the invention.

FIG. 1. is a perspective view of a human heart showing one embodiment of the current invention.

FIGS. 2A and 2B are perspective views of one embodiment of a mechanism to put the elastic connectors under tension, with reference to the events of the natural cardiac cycle.

FIG. 3 and FIG. 4 are perspective views of another embodiment of a mechanism to put elastic connectors under tension, with reference to the events of the natural cardiac cycle

FIGS. 5A and 5B are perspective views of another embodiment of a mechanism to put elastic connectors under tension, with reference to the events of the natural cardiac cycle.

FIGS. 6A and 6B are perspective views of another embodiment of a mechanism to put elastic connectors under tension, with reference to the events of the natural cardiac cycle.

### DETAILED DESCRIPTION OF THE DRAWINGS

The present invention is best described by its relation to the natural human heart, and therefore the natural anatomy and function of the heart is explained in some detail.

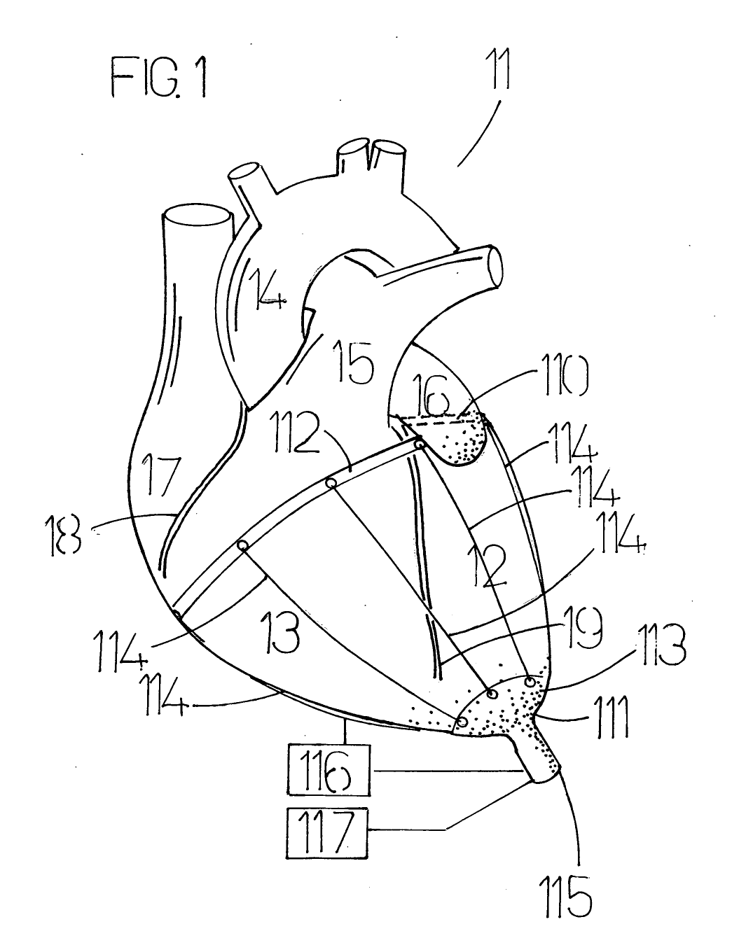

Referring to FIG. 1, a human heart is shown in perspective, together with an embodiment of the present invention in position. The heart 11 has two major pumping chambers, those being the left ventricle 12 and the right ventricle 13. The left ventricle pumps blood forwards into the aorta 14 and onwards throughout the body. The right ventricle pumps blood forwards into the pulmonary artery 15 and onwards through the lungs to be oxygenated. The aorta 14 and the pulmonary artery 15 are collectively known as the great vessels. The heart has two smaller chambers, the left atrium 16 and the right atrium 17, each of which is an entry chamber for blood into the respective ventricles. The heart muscle itself obtains its blood supply from the coronary arteries which originate from the aorta then coarse along the outer surface of the heart and give off branches to supply the heart muscle with blood. The three main coronary arteries are the right coronary artery 18, the left anterior descending coronary artery 19 and the circumflex coronary artery 110. The upper part of the heart is referred to as the base, the lower pole of the heart is known as the apex 111. The cardiac cycle refers to the changes in the shape and movement of the heart with each heart beat. Specifically the period of time during which the muscle in the walls of the ventricles contracts, causing the ventricles to eject their contents (blood) into the great vessels is known as systole. The period of time during which the ventricles are filling with blood again is known as diastole. The above described structure and function of the heart is known to those skilled in the art.

By way of non-limiting example, a possible embodiment of an invention to augment the contraction of the ventricles during systole is now discussed. A necessary component of the invention is a circumferential band 112, placed like a collar around the heart immediately proximate to the external surface of the natural heart. The current embodiment envisages this band being at approximately the level of the groove between the atria and the ventricles, but this need not be the case. This band is anchored securely into the actual structure of the natural heart at this position. By way of non-limiting examples, the circumferential band may be anchored in this position by surgical suture or chemical adhesive.

A second element of the present embodiment is a cap positioned on the apex of the heart, and this would be described as the apical cap 113. The apical cap is anchored securely into the actual structure of the natural heart, immediately proximate to the external surface of the heart at this position. By way of non-limiting examples, the apical cap may be anchored in this position by surgical suture or chemical adhesive.

A number of connectors 114 join the circumferential band to the apical cap. The exact number of these connectors is not vital, but would likely number at least 5 in any iteration of the invention. The connectors are positioned in a longitudinal pattern from the circumferential band 112 to the apical cap 113. A likely property of the connectors 114 in most iterations of the invention is elasticity, such that when the fibres are stretched they will tend to resist the stretch and will have the mechanical property of seeking to return to their original length. The effect of elasticity of the connecting bands 114 will be to apply forces that try to pull the apical cap 113 and the circumferential band 112 together. Because both the apical cap 113 and the circumferential band 114 are anchored into the structure of the natural heart, this will have the effect of diminishing the interior volumes of the ventricles and hence augment the expulsion of the ventricular contents. By this mechanism, the cardiac output will be increased, which is the purpose of this invention. A mechanical device 115 is required for this invention to have the desired effect. The mechanical device 115 needs to have the effect of stretching the connecting bands 114 repeatedly, in time with the natural contraction of the heart (systole), while removing the forces of elastic stretch upon the connecting bands 114 during diastole. Synchronization of the timing of the alternate stretching and relaxation of the connecting bands 114 with the natural events of the cardiac cycle is of utmost importance. During diastole, the ventricular filling phase, the connecting bands 114 need to be unstretched so that they do not inhibit filling of the ventricle. At the onset of systole, the connecting bands 114 will need to be rapidly stretched, preferably within 40 milliseconds, to put the connecting bands 114 under tension, and stay in the stretched conformation until cardiac contraction is finished. At the onset of ventricular filling the apical device needs to rapidly shorten again. All of the timing is possible by reference to the electrocardiogram (ECG), which is the intrinsic electrical activity of the heart with each heart beat cycle.

A component with the ability to detect the intrinsic activity of the heart is required 116, and has the purpose of activating the mechanical mechanism to stretch the connectors 114 at the correct timing in relation to the activity of the heart. In one iteration of the invention, the component to detect intrinsic activity of the heart 116 may be a custom modification of a pacemaker device.

A source of energy 117 is required to provide power for the mechanism 115 which stretches the connecting bands 114. By way of non-limiting examples of the energy source 117 this may be an electrical battery, or a mechanical source of energy, or a hydraulic source of energy. In one iteration of the invention, the energy source 117 may also provide power to allow the component that detects intrinsic activity of the heart 116 to perform its function.

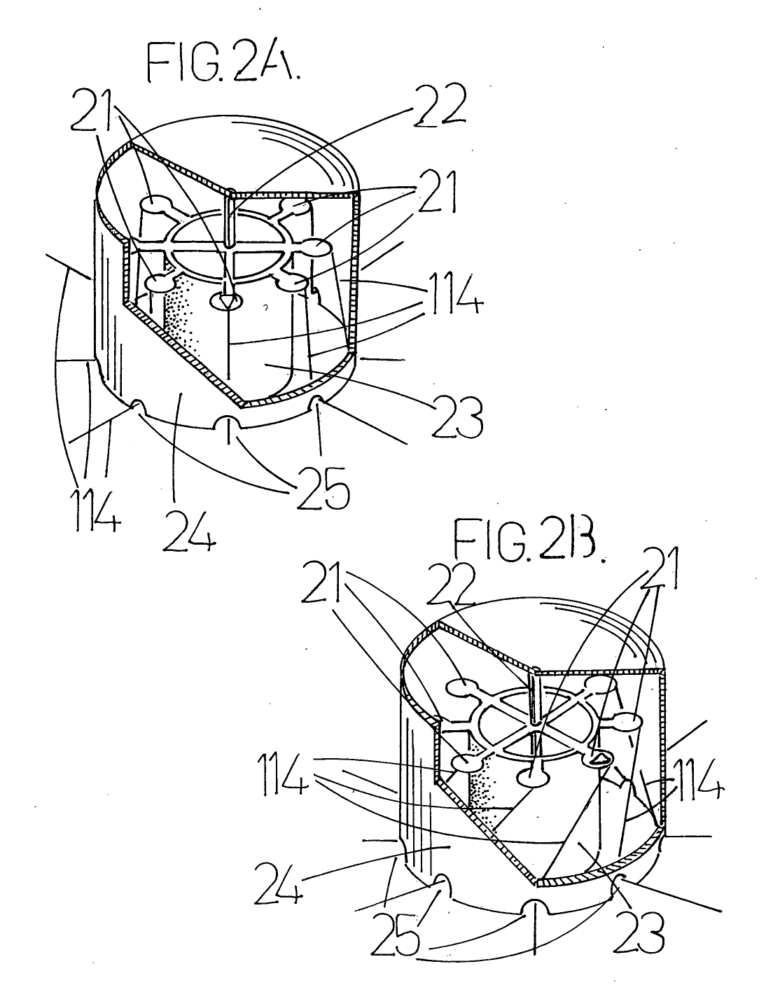

Referring to FIG. 2A and FIG. 2B, perspective views are shown of one non-limiting embodiment of a mechanism to put the elastic connectors 114 under intermittent tension, with reference to the events of the natural cardiac cycle. Specifically FIG. 2A, demonstrates the embodiment during the diastole (relaxation) phase of the natural cardiac cycle, and FIG. 2B demonstrates the embodiment during the systole (contraction) phase of the natural cardiac cycle. The elastic connectors 114, are each connected to a spoke 21. Each spoke 21 radiates from a central axle 22. The axle is rotated by a motor 23, which in turn causes the spokes to rotate. The spokes 21 and motor 23, are contained in an outer housing 24 which allows the rotation to occur without being inhibited by surrounding organs or tissues of the body. The housing has apertures 25, to allow the elastic connectors 114 to exit the housing, and also has the effect of redirecting the elastic connectors 114 in various directions in close proximity to the outer surface of the heart as shown in FIG. 1. In the views shown, the spokes 21 have rotated 45° around the axle 22 from their position in FIG. 2A to their position in FIG. 2B. This has the effect that the distance from the tip of the spoke 21 to the aperture 25 is longer in FIG. 2B than in FIG. 2A, thereby placing each elastic connector 114 under stretch.

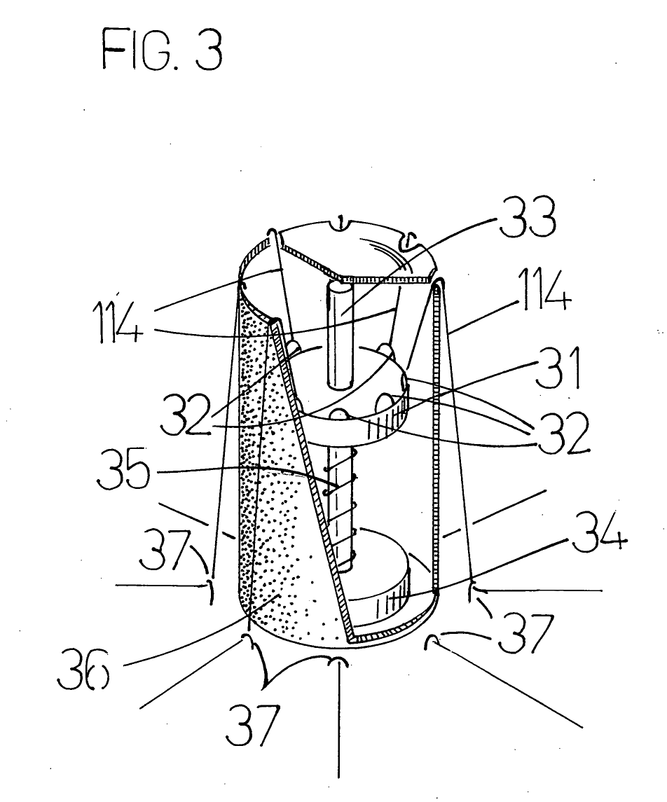

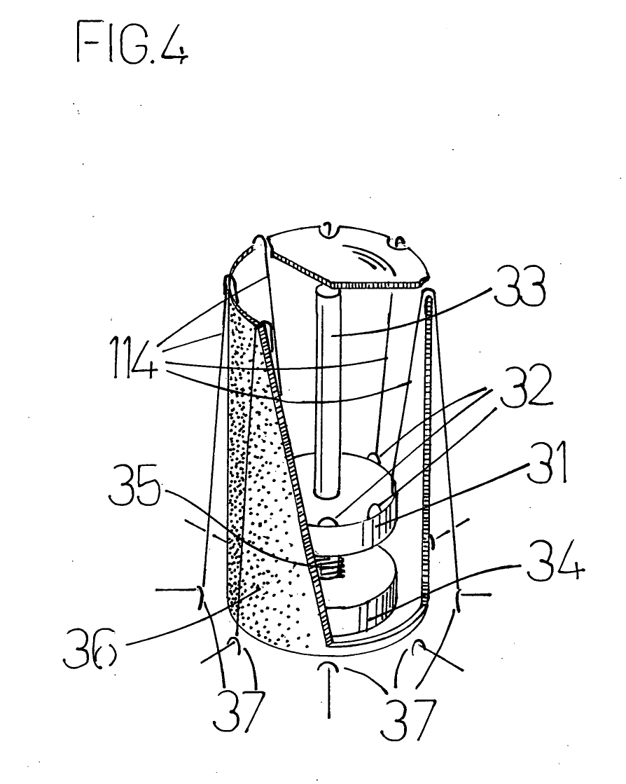

Referring to FIG. 3 and FIG. 4, perspective views are shown of another non-limiting embodiment of a mechanism to put the elastic connectors 114 under intermittent tension, with reference to the events of the natural cardiac cycle. Specifically FIG. 3 demonstrates the embodiment during the diastole (relaxation) phase of the natural cardiac cycle, and FIG. 4 demonstrates the embodiment during the systole (contraction) phase of the natural cardiac cycle. The elastic connectors 114, are each connected to a central piston 31 at one or more points 32. The piston may, in one embodiment, be able to slide freely upon an axle 33. There is a base component 34 firmly attached to the lower end of the said axle 33. A spring 35 is coiled around the axle between the base component 34 and the piston 31, and this spring tends to act to push the piston 31 away from the base component 34. FIG. 3 demonstrates the resting state of the iteration. In FIG. 4 a mechanism has been activated to draw the piston 31 and the base component 34 together. By way of non-limiting example, this mechanism may be magnetic attraction between the piston 31 and the base component 34. This movement has the effect of placing the elastic connectors 114 under stretch by increasing their length. When the mechanism that is causing attraction of the piston 31 toward the base component 34 is stopped, the action of the spring 35, returns the mechanism to its resting state once more, as shown in FIG. 3. The described mechanism is contained within an outer housing 36 so that the action can occur without being inhibited by surrounding organs or tissues of the body. A number of eyelets 37 serve to redirect the elastic connectors 114 in various directions in close proximity to the outer surface of the heart as shown in FIG. 1.

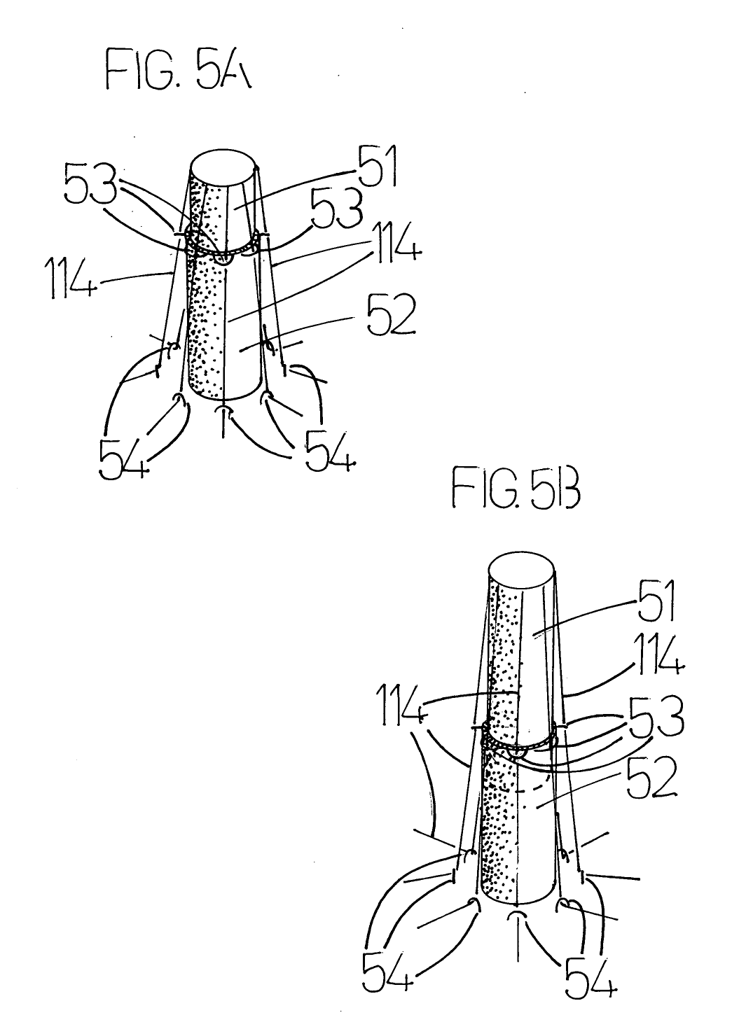

Referring to FIG. 5A and FIG. 5B, perspective views are shown of another non-limiting embodiment of a mechanism to put the elastic connectors 114 under intermittent tension, with reference to the events of the natural cardiac cycle. Specifically FIG. 5A demonstrates the embodiment during the diastole (relaxation) phase of the natural cardiac cycle, and FIG. 5B demonstrates the embodiment during the systole (contraction) phase of the natural cardiac cycle. The elastic connectors 114 are each connected to a piston 51. The piston can move within a chamber 52. FIG. 5A demonstrates the resting phase of the iteration. In FIG. 5B, a mechanism has extended the piston 51 out of the chamber 52. This has the effect of lengthening the elastic connectors 114 when the piston 51 is in the position shown in FIG. 5B, thereby stretching said connectors. There are sets of eyelets around the top edge of the chamber 53, and around its base 54, which have the combined effect to redirect the elastic connectors 114 in various directions in close proximity to the outer surface of the heart as shown in FIG. 1.

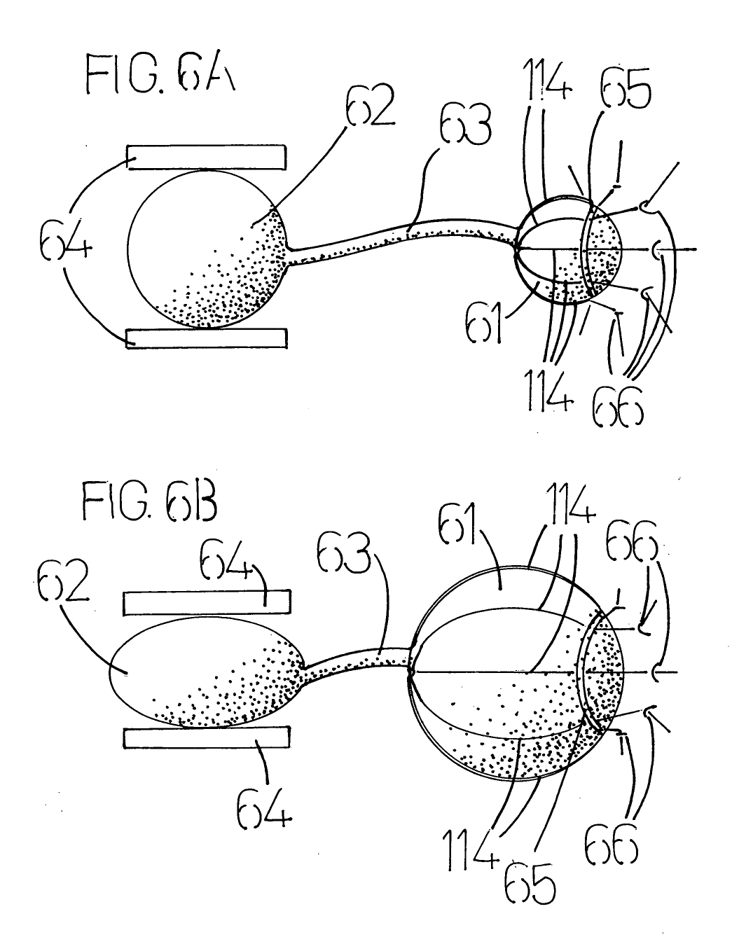

Referring to FIG. 6A and FIG. 6B, perspective views are shown of another non-limiting embodiment of a mechanism to put the elastic connectors 114 under intermittent tension, with reference to the events of the natural cardiac cycle. Specifically FIG. 6A demonstrates the embodiment during the diastole (relaxation) phase of the natural cardiac cycle, and FIG. 6B demonstrates the embodiment during the systole (contraction) phase of the natural cardiac cycle. In this embodiment the connectors are mounted longitundinally around a bladder 61 which contains gas or liquid. The bladder is connected to a second bladder 62, by hollow tubing 63. The second bladder 62 and the tubing 63 are also filled with gas or liquid in continuity with the contents of the main bladder 61. A mechanical component 64 acts to compress the second bladder 62, as demonstrated in FIG. 6B. The effect of this action is to move gas or liquid from the second bladder 62 into the main bladder 61, thereby expanding it. Expansion of the main bladder 61, in turn causes lengthening and stretching of the elastic connectors 114. A circumferential belt 65 around the main bladder, and a set of eyelets 66, have the effect of redirecting the elastic connectors 114 in various directions in close proximity to the outer surface of the heart as shown in FIG. 1.

### REFERENCE NUMERALS USED IN THE DRAWINGS

11. The heart
12. Left ventricle
13. Right ventricle
14. Aorta
15. Pulmonary artery
16. Left atrium
17. Right atrium
18. Right coronary artery
19. Left anterior descending coronary artery
110. Circumflex coronary artery
111. Cardiac apex
112. Circumferential band
113. Apical cap
114. Elastic connectors
115. Mechanical device to apply stretch to the elastic connectors
116. Component to detect intrinsic activity of the heart
117. Energy source
21. Spokes
22. Axle
23. Motor
24. Housing
25. Apertures
31. Piston
32. Connection points
33. Axle
34. Base
35. Spring
36. Outer housing
37. Eyelets
51. Piston
52. Chamber
53. Eyelets at top edge of chamber
54. Eyelets around the base
61. Bladder
62. Second bladder
63. Connecting tubing
64. Mechanical component to compress second bladder
65. Circumferential belt
66. Eyelets

### DETAILED DESCRIPTION OF THE INVENTION

This embodiment augments the pumping function of the heart. One component of the embodiment is a set of 5 or more connectors with elastic properties, located immediately proximate to the outer surface of the heart. Each of the elastic connectors is attached at one end to a circumferential band firmly attached to the heart, and at the other end to a component attached firmly to the heart at the anatomical apex of the heart. A mechanism exists to place the elastic connectors alternatively under tension (stretch) then being allowed to elongate (relax). When the elastic connectors are stretched they will have the action of drawing the apex and circumferential band together because the connectors are attached to said two components at either end. This results in external compression of the heart. This movement will cause the internal volume of the cardiac ventricles to be reduced, thereby encouraging expulsion of the blood contents of the ventricles to be expelled. This compressive action is carefully be timed so as to augment the natural cardiac contraction.

An important component of this embodiment is a mechanism to cyclically place the elastic connectors under stretch in time with the natural cardiac contraction. The stretching of the connectors must take place during the contraction phase of the normal cardiac cycle (known as systole). The precise mechanism or location of this component is not critical to its function. The critical aspect of this component is its ability to shorten (stretch) and lengthen the elastic connectors. In one iteration of the invention, an axle rotates forward and backward within a housing. In this iteration, the rotational movement of the axle is converted to stretching and relaxation of the connectors because the connectors are attached to the end of spokes that radiate from the rotating axle. In this way the connectors are stretched and lengthened alternately. In another iteration of the invention, the stretching mechanism consists of piston-like motion of a component, possibly by intermittent activation of a magnetic force. In this iteration, the elastic connectors are attached to the piston and the up and down movement of the piston is thereby converted to stretching and relaxation of the connectors. In a third iteration of the invention, there is intrinsically attached to the apical cap component a mechanism that extends and retracts, with the connectors attached to this mechanism. In this way the connectors are stretched and relaxed alternately. In a fourth iteration of this invention a bladder is intermittently filled with liquid or air thus expanding in size. The elastic connectors are closely applied to the outside of the bladder, therefore its expansion is converted into stretching of the connectors. These described iterations are not designed to be limiting to the scope of the description of this invention. Other mechanical solutions may exist to fulfil the stretching and elongating function of this component. The important concept is that there be a mechanism to stretch the longitudinal elastic connectors, in time with the natural cardiac contraction.

This iteration will contain a component to detect the intrinsic activity of the heart, and will utilize this information to activate the mechanism to stretch the elastic fibres at the correct time (early systole) and activate the mechanism to allow the connectors to shorten during cardiac relaxation (known as diastole.) In one non-limiting iteration, this component may be a custom modified electronic detector of the electrical activity of the heart.

Another component of this iteration is a source of energy to provide power to allow the mechanical component to perform its action of stretching, then allowing relaxation of the elastic connectors. In the most likely iteration, this energy source will be a battery. The location of this battery is not critical to the function of the invention. A battery fully implanted within the body is the preferred embodiment, and may include the capability for trans-cutaneous recharging of the battery by an external power source. In another iteration of this invention, the battery may be external to the body which would require electrical cables to penetrate the skin to allow the current to be conducted to the device (this iteration is less preferable). In another iteration, the energy may be in the form of mechanical or hydraulic energy that intermittently expands a fluid or air filled bladder. These described iterations are not designed to be limiting to the scope of the description of this invention. Other sources of energy may exist to fulfil the requirement of allowing the stretching and elongating mechanism to function.

1. A method to augment contraction of a weakened natural heart, comprising:
a. a circumferential anchor component attached firmly to the heart, in close proximity to the external surface of the heart, at or close to the anatomical feature known as the atrio-ventricular groove,
b. a cardiac cap component, firmly attached to the heart, in close proximity to the external surface of the heart, located at the anatomical apex of the heart,
c. a set of connectors, each with elastic properties, each of which attaches to the circumferential anchor at one end and to the cardiac cap located at the apex of the heart at the other end,
d. a mechanical means to alternatively stretch then relax said connectors, with the stretch part of the cycle timed so as to be synchronous with the heart's natural contraction,
e. a component to detect the intrinsic rhythm of the heart and activate the said mechanical mechanism at the appropriate time in relation to the heart's natural rhythm, and
f. an energy supply to provide the power to operate said mechanical means,
whereby the said method will augment the expulsion of the blood contents of the heart ventricles due to the tendency of said circumferential anchor and said cardiac cap to be drawn closer together, thereby reducing the internal volume of the cardiac ventricles, at the same time as the heart's natural contraction.

2. The mechanical means of claim 1 wherein said mechanical means is a method to cause rotational motion of an axle to be converted into alternating stretching and relaxation linear motion of the said elastic connectors of claim 1

3. The mechanical means of claim 1 wherein said mechanical means is a method to cause piston-like movement of an inner component within an outer casing to be converted into alternating stretching and relaxation linear motion of said elastic connectors of claim 1

4. The mechanical means of claim 1 wherein said mechanical means is a method to cause inflation and deflation of a bladder to cause alternating stretching and relaxation linear motions of said elastic connectors of claim 1

5. The energy supply of claim 1 wherein said energy supply is an electrical battery fully implanted within the human body, which may include the capability of trans-cutaneous recharging by an external energy source without the need to breach the skin

6. The energy supply of claim 1 wherein said energy supply is an electrical battery external to the body with electrical wires penetrating the skin.

7. The energy supply of claim 1 wherein said energy supply is the movement of fluid from one bladder to another in the form of hydraulic energy.
